(19) United States
(12) Patent Application Publication
(10) Pub. No.: US 2008/0026374 A1
(43) Pub. Date: Jan. 31, 2008

(54) Compositions and Methods for Isolation of Biological Molecules

(75) Inventors: Fuqiang Chen, St. Louis, MO (US); Carol Kreader, St. Louis, MO (US)

Correspondence Address: SENNIGER POWERS (SGM), ONE METROPOLITAN SQUARE, 16TH FLOOR, ST. LOUIS, MO, 63102, US

(73) Assignee: SIGMA ALDRICH CO., St. Louis, MO (US)

(21) Appl. No.: 11/461,263

(22) Filed: Jul. 31, 2006

(51) Int. Cl. C12Q 1/68; C12M 3/00

(52) U.S. Cl. 435/6; 435/287.2

(57) ABSTRACT

The present invention provides charged separation media, kits comprising the separation media, and methods of using the same to bind and optionally isolate biological molecules. The charged separation media and methods utilize a polyion non-covalently bound to the separation media to bind the desired biological molecule to the separation media.

### FIELD OF THE INVENTION

The present invention generally relates to the binding and isolation of biological molecules such as nucleic acids and proteins.

### BACKGROUND OF THE INVENTION

Ion chromatography has become a valuable tool for separating desired biological materials from samples containing additional, undesired materials. It is generally used to isolate specific target materials such as enzymes, hormones, proteins, and nucleic acids on the basis of the specific charge interactions between the target material and an immobilized moiety. Generally, a support comprising the immobilized moiety is contacted with a binding solution comprising the target material. Once the target is bound, the support may be washed to remove any undesired biological materials or impurities. Thereafter, the target may be eluted from the immobilized moiety or the target/moiety complex may be eluted from the support, thereby providing a solution containing the desired target material in the absence of the undesired biological material originally present in the sample.

The binding and elution of target molecules according to this method often involves the use of stringent binding, wash, or elution conditions. Typically, these conditions, and in particular the elution conditions, require the use of solutions comprising high concentrations of salts that must subsequently be removed from the eluted substance in order to further process or use the target compound. The requirement of salt addition and removal unnecessarily increases processing time and costs and can pose constraints on high throughput and automation procedures.

### SUMMARY OF THE INVENTION

Among the various aspects of the present invention, is a method of binding and optionally isolating a biological molecule using a separation media charged with a polyion wherein the polyion is non-covalently bound to the separation media and can be removed from both the separation media and the biological molecule under mild ionic conditions.

Briefly, therefore, one aspect of the present invention is a method of binding and optionally isolating a biological molecule from a mixture. The method comprises combining the mixture with a separation media having a polyion non-covalently bound to the surface thereof to bind the biological molecule to the separation media. The method may also comprise washing the charged separation media with a wash solution to remove the polyion from both the separation media and the biological molecule and thereafter eluting the biological molecule from the separation media with an elution solution.

Another aspect of the present invention is a charged separation media for use in the binding and optional isolation of a biological molecule from a mixture. The charged separation media comprises a polyion non-covalently bound thereto, wherein the polyion is a polyion other than a nucleic acid or a naturally occurring protein.

Yet another aspect of the present invention is a kit for binding and optionally isolating a biological molecule from an aqueous mixture. The kit comprises a separation media, a polyion, and instructions, wherein the polyion is supported or supportable by the separation media by non-covalent bonding and the instructions provide directions for the use of the separation media charged with the polyion, to bind the biological molecule.

Other objects and features will be in part apparent and in part pointed out hereinafter.

### BRIEF DESCRIPTION OF THE DRAWINGS

FIG. 1 depicts a gel demonstrating the binding of plasmid DNA to a negatively charged separation media through the polycation linkage of spermine according to the methods described herein. Binding columns comprised a mini spin basket containing a layer of the Biodyne C membrane. Each column was charged with either a 0.5 M spermine solution or water. Column 1 represents a 1-kb DNA ladder (Sigma Product Number D0428). Column 2 represents the input pSPORT-βgal plasmid DNA sample in 100% proportion (control). Columns 3 to 7 represent the column flow-through samples after forcing the plasmid DNA solution through the columns by centrifugation. Columns 3 to 5 represent the columns charged with spermine. Columns 6 and 7 represent the columns charged with water. Columns 8 and 9 represent the recovered plasmid DNA samples from two of the spermine-charged columns after the columns were washed with 200 μl of a wash solution (25 mM EDTA, 300 mM NaCl, 60% isopropanol) and eluted with 100 μl of a low salt buffer (10 mM tris, 1 mM EDTA, pH 8.0). Plasmid DNA was bound to the columns charged with spermine and quantitatively recovered after the polycation was removed from the separation media by the wash solution.

FIG. 2A depicts a gel demonstrating the purification of PCR samples according to the methods described herein. PCR samples were isolated on silica columns charged with varying concentrations of spermine. Column 1 in each group represents the input PCR sample at 100% proportion (control). Column 2 in each group represents PCR products purified with silica columns charged with a 0.5M spermine solution. Column 3 in each group represents PCR products purified with silica columns charged with a 1M spermine solution. Group 1 represents a 143 bp corn Adh fragment amplified by Taq. Group 2 represents a 350 bp Lambda DNA fragment amplified by Taq. Group 3 represents a 643 bp corn Adh fragment amplified by Taq. Group 4 represents the 643 bp corn Adh fragment amplified by RedTaq®. PCR fragments greater than 143 bp were quantitatively recovered and primer dimmers of about 50 bp were completely eliminated.

FIG. 2B depicts a gel demonstrating the purification of PCR samples according to the methods described herein. PCR samples were isolated on silica columns charged with varying concentrations of spermine. Column 1 in each group represents the input PCR sample at 100% proportion (control). Column 2 in each group represents PCR products purified with silica columns charged with a 0.5M spermine solution. Column 3 in each group represents PCR products purified with silica columns charged with a 1M spermine solution. Group 1 represents a 1.5 kb Bacterial Alkaline Phosphatase (BAP) fragment amplified by AccuTaq™. Group 2 represents the 1.5 kb BAP fragment amplified by RedTaq®.

FIG. 3 depicts a gel demonstrating the purification of different sizes of plasmid DNA according to the methods described herein. Plasmid DNA was isolated from the lysate on a silica column charged with spermine. Columns 1 and 19 represent a 1-kb DNA ladder (Sigma Product Number D0428). Columns 2 and 18 represent 140 ng and 70 ng, respectively, of pSPORT-βgal plasmid DNA purified by a conventional method (GenElute™ Endotoxin-free Plasmid Maxiprep Kit). Columns 3 to 17 represent 1 μl each of 15 plasmid DNA samples purified according to the methods described herein. Columns 3 to 6 represent four pSPORT-βgal plasmid DNA (7.9 kb) samples each purified from 400 μl of lysate. Columns 7 to 10 represent four pSPORT-βgal plasmid DNA samples each purified from 800 μl of lysate. Columns 11 to 14 represent four Bluescript plasmid DNA samples (3.0 kb) purified from 400 μl of lysate. Columns 15 to 17 represent four Bluescript plasmid DNA samples purified from 800 μl of lysate.

FIG. 4 depicts a non-denaturing agarose gel demonstrating the capture and recovery of total RNA samples according to the methods described herein. RNA samples were captured on four different silica columns charged with a 0.5M spermine solution. The first and last columns represent a 1-kb DNA ladder (Sigma Product Number D0428). Column C1 represents RNA recovered in the first or second elution or RNA in the flow through fraction using 2 layers of Osmonics glass filter paper G15. Column C2 represents RNA recovered or in the flow through fraction from 1 layer G15 and 1 layer of Ahlstrom glass filter paper Grade 151. Column C3 represents RNA recovered or in the flow through fraction from 1 layer G15 and 1 layer of Ahlstrom glass filter paper Grade 121. Column C4 represents RNA recovered or in the flow through fraction from 1 layer of Grade G151 and 1 layer of Grade 121.

### DETAILED DESCRIPTION OF THE INVENTION

The present invention is generally directed to an improved solid separation media or medium useful for the binding and isolation of biological molecules such as nucleic acids and proteins and to methods of using such separation media to bind and isolate such biological molecules.

One aspect of the present invention, therefore, is directed to a charged separation media useful for the binding and optional isolation of a biological molecule from a sample containing the same. The charged separation media generally comprises a charged polyion non-covalently bound to the separation media. The charged polyion is used to bind a desired biological molecule (sometimes referred to herein as a target molecule) from a sample containing the molecule. The biological molecule may thereafter be subject to additional analysis, study, or use. The polyion bound to the separation media will generally be different from the biological molecule, interact with the biological molecule based on charge interactions, have a molecular weight within a particular range, be capable of being removed from both the separation media and the biological molecule under mild conditions, or not be a nucleic acid or a naturally occurring protein. After being bound, the biological molecule may be isolated by washing the column to remove the polyion from both the separation media and the biological molecule and to remove impurities (i.e., materials other than the desired biological molecule) from the separation media. Advantageously, thereafter the biological molecule may be eluted from the separation media under very mild conditions.

Another aspect of the present invention is directed to a method of binding and optionally isolating a biological molecule, such as, for example, a nucleic acid, polynucleotide, protein, or polypeptide, from a sample. The method typically involves the use of a charged separation media as described above and generally comprises the steps of charging a solid separation media by contacting the separation media with a polyion and thereafter binding an oppositely charged biological molecule to the polyion. Once bound, the biological molecule may be optionally isolated by washing the charged separation media with a wash solution generally comprising an alcohol and a salt to remove (desorb) the polyion from both the separation media and the charged biological molecule and then eluting the charged biological molecule from the separation media with an elution solution generally comprising water, a buffer, or a mild salt solution.

The charged separation media and methods of the present invention may be used to bind and isolate any of a number of different biological molecules, including, for example, bioparticles, such as, for example, cellular structures, membrane proteins, viral vectors, and viruses; nucleic acids, such as, for example, DNA, RNA, siRNA, miRNA, and plasmids; modified nucleic acids, such as, for example, peptide nucleic acids (PNA) and locked nucleic acids (LNA); nucleotides, oligonucleotides, and polynucleotides; peptides, including oligopeptides and polypeptides; and proteins, such as, for example, antibodies, antigens, enzymes, hormones, and immunoglobulins.

Unique to the charged separation media and the methods of the present invention is the use of a non-covalently bound polyion to charge the separation media and to bind the desired biological molecule to the separation media. The polyion is non-covalently bound to the separation media and forms a bridge or link between the separation media and the biological molecule that may be removed in order to isolate the biological molecule. Generally, some or all of this process may be accomplished under mild conditions. Such mild conditions typically do not require the use of binding solutions or of elution solutions containing a high concentration of salt that must be subsequently removed from the eluted biological molecule by additional procedures. In particular, because of the use of a non-covalently bound polyion to bind the biological molecule, the biological molecule may be bound in the absence of a binding solution or without adjusting the binding conditions. Moreover, the biological molecule may be eluted using a low ionic strength or nonionic elution solution, thereby allowing for the subsequent use or analysis of the isolated biological molecule in the absence of additional processing steps. Because additional processing of the isolated biological molecules is not required to remove large amounts of salts or to otherwise lessen the stringent conditions created by typical binding and isolation procedures, the overall processing constraints, time, and costs associated with other known methods are reduced. In addition to these advantages, the methods of the present invention are safer and more adaptable to high throughput automation.

Separation Media

Any number of solid separation media may be used in accordance with the present invention. Typically, such separation media will be capable of being charged or loaded with a polyion. Charging or loading the separation media in the manners described below typically results in the polyion being non-covalently bound to the separation media, thereby allowing the separation media to have a desired positive or negative charge and to bind negatively or positively charged biological molecules, respectively.

Generally, solid separation media suitable for use according to the present invention include solid separation media comprising an insoluble and usually rigid matrix or stationary phase that is capable of forming non-covalent bonds with a polyion. The separation media will generally have a sufficient surface area to permit adequate binding capacity (number of binding sites) and parking area (distance between binding sites) for non-covalent binding of the polyion. Moreover, the separation media should be capable of being separated from a solute mixture comprising the biological molecule and, typically, at least one contaminant after the biological molecule has been combined therewith.

The separation media may be either charged (positively or negatively) or neutral prior to being contacted with the polyion, as the charge of the separation media is subsequently manipulated by the addition of the polyion. By way of example, the separation media, as well as the polyion, may be tailored for binding of the desired target molecule. Accordingly, a separation media that possesses a neutral or negative native charge may be used to bind or isolate a negatively charged biological molecule by charging the column with a polycation before contacting the column with the biological molecule. Similarly, a separation media that possess a neutral or positive native charge may be used to bind or isolate a positively charged biological molecule by charging the column with a polyanion before contacting the column with the biological molecule.

Exemplary separation media include stationary phases or matrices in liquid chromatography (LC), high pressure liquid chromatography (HPLC), including particulate matrices embedded into or bound to filters and magnetic or non-magnetic porous matrix particles, silica gels, silica magnetic particles, glass particles (including, quartz, vitreous, silica, controlled pore particles, and glass fibers), chelating matrices, and anion and cation exchange matrices or surfaces, such as, for example, walls of tubes, plate wells, or pipette tips, coated with any of the above. Preferred separation media include chelating matrices, cation exchange matrices, anion exchange matrices, and silica matrices, including chelating, cation exchange, anion exchange, and silica resins, filters, beads, membranes, and coated surfaces.

Separation media of particular interest are those that possess or carry a functional group capable of non-covalently binding to or being bound by a polyion. Examples of such functional groups commonly found on separation media include, for example, iminodiacetic acid (IDA) functional groups (Sepharose 6B Fast Flow Resin (Sigma-Aldrich Co., St. Louis, Mo.); Chelex 100 (Biorad, Hercules, Calif.); immobilized iminodiacetic acid (Pierce Biotechnology, Inc., Rockford, Ill.), and iminodiacetic acid agarose (Sigma-Aldrich, St. Louis, Mo.)); nitrilotriacetic acid (NTA) functional groups; carboxylic acid functional groups; sulphonic acid functional groups; and amine functional groups.

Of particular interest are separation media that form a relatively weak association with the polyion. Ideally, the separation media will form an association with the polyion that is sufficiently strong to allow the polyion to remain bound to the separation media until the separation media is contacted with a substance intended to remove the polyion, but sufficiently weak such that the polyion may be easily and efficiently removed from the separation media by a substance, such as, for example, an ionic solution, and in particular an ionic solution of moderate to low ionic strength, that is intended to be used to remove the polyion. Moreover, preferred separation media will possess a high binding capacity, filtration depth, and flow characteristics. A preferred separation media, therefore, is a silica separation media. The silica separation media may be in the form of silica beads, silica matrices, silica resins, silica filters, or silica membranes. A particularly preferred separation media is silica filters.

Selection of a Polyion

The solid separation media may be charged with either a positive (polycation) or negative (polyanion) polyion. The choice of polyion is dependent upon several factors, including the charge of the biological molecule to be isolated, the charge of the separation media prior to contacting the same with the polyion, the number of free charges on the surface of the separation media that are available to bind the polyion, the number of free charges on the polyion available for binding either or both the separation media and the biological molecule, and the strength of the non-covalent bond formed between the separation media and the polyion.

If the biological molecule is a negatively charged molecule or polymer, such as, for example, a nucleic acid or a polynucleotide, acidic proteins, polypeptides or proteins at a pH above their pKa, fatty acids, acidic vitamins, acidic growth hormones, and endotoxins, the solid separation media may be charged with a polycation, such as, for example, spermine, spermidine, putrescine, polylysine, polyarginine, or polyhistidine. Alternatively, if the biological molecule is a positively charged molecule or polymer, such as, for example, basic proteins or polypeptides, proteins or polypeptides at a pH below their pKa, polyamines, alkaloids, or basic growth hormones, the solid separation media may be charged with a polyanion, such as, for example, citric acid, succinic acid, polyacrylic acid, polyglutamic acid (polyglutamate), polyaspartic acid (polyaspartate), and heparin sulfate.

Because the charges of the polyion and the biological molecule are generally opposite of one another, the polyion that is used to charge the separation media will typically be a substance or composition that is of opposite charge and/or is not the biological molecule to be bound and isolated using the charged column. Accordingly, the polyion will generally not be a nucleic acid, an oligonucleotide, or a polynucleotide, such as, for example, DNA or RNA, particularly when attempting to isolate and purify a biological molecule that comprises the same. Likewise, the polyion will generally not be a peptide, an oligopeptide, a polypeptide, or a protein, and in particular a naturally occurring protein, particularly when attempting to bind and isolate a biological molecule that comprises the same. The polyion and the biological molecule may, however, be the same type of molecule in instances where the charge of one is the opposite of the other. By way of example, the polyion may be a peptide, an oligopeptide, a polypeptide, or a protein, such as for example, polylysine, if the biological molecule were to be a peptide, an oligopeptide, a polypeptide, or a protein having an opposite charge, such as, for example, an acidic protein. Likewise, the polyion may be a peptide, an oligopeptide, a polypeptide, or a protein, such as, for example, polyaspartic acid, if the biological molecule were to be a peptide, an oligopeptide, a polypeptide, or a protein having an opposite charge, such as, for example a basic protein.

The choice of a polyion is also affected by the charge of the separation media prior to contacting the separation media with the polyion (i.e., the native charge of the separation media). Specifically a separation media that possesses a neutral or negative native charge may be used to isolate a negatively charged biological molecule by charging the media with a polycation before contacting the media with the biological molecule. Similarly, a separation media that possess a neutral or positive native charge may be used to isolate a positively charged biological molecule by charging the media with a polyanion before contacting the media with the biological molecule.

The choice of a polyion is also affected by the number of free charges of one or more functional groups or binding sites located or contained on the surface of the separation media that are available to bind the polyion. The greater the number of free charges on a functional group(s) or binding site(s), the greater the number of free charges on the polyion that may be bound to or bound by the functional group(s) or binding site(s) (and ultimately, therefore, the separation media), and therefore, a decrease in the number of free charges on the polyion that may be available to bind the biological molecule. Accordingly, the polyion selected to charge the separation media will generally have at least one more free charge than the number of free charges of a functional group or binding site contained on the separation media. By way of example, some sepharose supports contain an iminodiacetic acid (IDA) functional group on the surface of the support (for example, Sepharose 6B Fast Flow Resin (Sigma-Aldrich Co., St. Louis, Mo.); Chelex 100 (Biorad, Hercules, Calif.); Immobilized Iminodiacetic Acid (Pierce Biotechnology, Inc., Rockford, Ill.), and Iminodiacetic Acid Agarose (Sigma-Aldrich Co., St. Louis, Mo.)). A single IDA functional group possesses two negative charges with which to non-covalently bind a polyion. In such an example, a polyion having three or more free positive charges may be selected for use in this instance, as two of the positive charges will be used to non-covalently bind one, or possibly more, IDA functional group(s), leaving one or more positive charges to bind a negatively charged biological molecule. The greater the number of free charges possessed by the polyion, the more tightly the polyion will bind the biological molecule or the greater the number of biological molecules that may be bound.

Examples of functional groups that may be used to bind polycations include for example, iminodiacetic acid functional groups, nitrilotriacetic acid functional groups, sulfonic acid functional groups, carboxylic acid, including di- and tri-carboxylic acid, functional groups, aspartic acid functional groups, tris(carboxymethyl)ethylenediamine (TED) functional groups, silicate functional groups, and ethylenediaminetetraacetic acid functional groups.

Examples of functional groups that may be used to bind polyanions include for example, primary, secondary, tertiary, and quaternary amine functional groups.

A related factor affecting the selection of a polyion is the number of free charges on the polyion available for binding the separation media and/or the biological molecule. As discussed above, for each free charge of the polyion bound by a functional group or binding site on the separation media, there is one less free charge of the polyion to be bound by a biological molecule. The greater the number of free charges possessed by the polyion, the more tightly the polyion will bind the biological molecule or the greater the number of biological molecules that may be bound.

As such, the polyion will generally possess at least about one more free charge than a functional group or binding site on the separation media, preferably at least about two more free charges, more preferably at least about three more free charges, even more preferably at least about four more free charges, and still more preferably at least about five more free charges. In a preferred embodiment, the polyion possesses about one more free charge than a functional group or binding site on the separation media. In a more preferred embodiment, the polyion possesses about two more free charges than a functional group or binding site on the separation media. And in an especially preferred embodiment, the polyion possesses about three more free charges than a functional group or binding site on the separation media.

Another factor affecting the selection of a polyion is the strength of the non-covalent bond formed between the separation media and the polyion. While the bond should be sufficiently strong to maintain the association between the separation media and the polyion in the absence of a wash solution, it should also be sufficiently weak to allow for easy and efficient removal of the polyion from both the separation media and the biological molecule in the presence of a wash solution, and preferably a wash solution of mild to low ionic strength. This factor may be affected not just by the selection of a particular polyion, but also by the choice of a separation media (i.e., whether the separation media is, for example, an exchange matrix, a chelating matrix, or a silica matrix).

Generally, a selected polyion will be able to be removed from both the separation media and the biological molecule by washing with a mild to low ionic strength solution. Such a polyion will generally disassociate from a separation media when not also bound to a biological molecule by washing the separation media with an ionic solution having an ion concentration of less than about 0.5M, preferably less than about 0.25M, and more preferably less than about 0.1M. Such a polyion will also generally dissociate from both a separation media and a biological molecule (when bound by a biological molecule) by washing the separation media with an ionic solution having an ion concentration of about 0.1M to about 3M, preferably from about 0.25M to about 2M, and more preferably from about 0.5M to about 1M.

Yet another factor affecting the selection of a polyion is the size of the polyion to be used. Typically, the polyion should be of a size sufficient to present a number of free charges that may be bound by the separation media or the desired biological molecule, but not so large that the polyion hinders its own removal from the separation media or the biological molecule as a result of, for example, high charge density. By way of example, nucleic acids, native proteins, and other large polyions are generally not preferred for this reason. Accordingly, the polyion used in accordance with the present invention will generally be of a molecular weight of about 100 Daltons to about 100,000 Daltons, preferably about 150 Daltons to about 50,000 Daltons, more preferably about 150 Daltons to about 25,000 Daltons, still more preferably about 150 Daltons to about 15,000 Daltons, and most preferably about 150 Daltons to about 1,000 Daltons. The size of the polyion may vary depending on the charge density and the number of charges on the polyion, the particular structural and functional characteristics of the polyion, and the type of biological molecule to be isolated.

Accordingly, common polyions that may be used in accordance with the present invention include, for example, polycations. Examples of suitable polycations include, for example, polyamines, including both natural and synthetic polyamines, such as for example, putrescine, spermine, and spermidine; cationic oligopeptides, such as, for example di-, tri-, and tetra-peptides; and cationic polypeptides, including, for example, polylysine, polyarginine, and polyhistidine. Common polyions that may be used in accordance with the present invention also include, for example, polyanions. Examples of suitable polyanions include, carboxylic acids (including di- and tri-carboxylic acids), such as, for example, citric acid, succinic acid, and polyacrylic acid; anionic oligopeptides, such as, for example, di-, tri-, and tetra-peptides; anionic polypeptides, such as, for example, polyglutamic acid (polyglutamate), and polyaspartic acid (polyaspartate); and polysaccharides, including, for example, heparin sulfate. Particularly preferred polyions include spermine, spermidine, putrescine, polyarginine, polylysine, and polyhistidine. Accordingly, in one embodiment, the separation media is charged by contacting the separation media with spermine. In another embodiment, the separation media is charged by contacting the separation media with spermidine. In yet another embodiment, the separation media is charged by contacting the separation media with putrescine. In still another embodiment, the separation media is charged by contacting the separation media with polyarginine. In another embodiment, the separation media is charged by contacting the separation media with polylysine. In yet another embodiment, the separation media is charged by contacting the separation media with polyhistidine.

Charging the Separation Media

A separation media of the present invention may be charged in a number of ways. Selection of the particular charging method is affected by factors such as the type of separation media being charged, the selected polyion, and the form of the polyion (i.e., whether the polyion is in solution). Generally, the separation media is charged by contacting the separation media with the selected polyion according to any of a number of well known methods, including for example, soaking the separation media in a solution containing the polyion, rinsing or washing the separation media with several units of a solution containing the polyion, or particularly in the case of filter membranes or glass wool, combining the polyion with the separation media and subsequently centrifuging or vacuum filtering the separation media to disperse the polyion throughout the same. Regardless of the method used, the polyion may charge the separation media by non-covalently attaching to the separation media.

Mixture Containing the Biological Molecule

Once the separation media is charged, the separation media is contacted with the biological molecule, thereby allowing the biological molecule to bind to or to be bound by the separation media via the polyion.

As described above, the biological molecule may be any of a number of substances that may be bound by the charged separation media. Generally, the biological molecule will be contained in a solution or mixture that may be contacted with the separation media. This solution or mixture may be, or may be combined with, a binding solution having a mild or low ionic strength to aid in the binding of the biological molecule to or by the separation media. Preferably, however, the solution or mixture is neither an ionic binding solution nor is it combined with an ionic binding solution, and the biological molecule is bound to or by the separation media in the absence of or without the aid of a binding solution.

In addition to the biological molecule, the mixture or solution will typically also contain undesired biological material (i.e., contaminants) from which the target biological molecule is to be isolated. The sample mixture or solution may also contain buffers, enzymes, and detergents. It may also contain cellular debris that remains from cell lysis or components or substances that remain from reactions used to obtain the biological molecule, such as, for example, PCR components. Alternatively, these extraneous components may also be removed from the sample mixture or solution prior to contacting the separation media with the mixture or solution.

Typically, the sample mixture or solution may have a pH of less than about 11, preferably less than about 10, more preferably from about 2 to about 9, still more preferably from about 5 to about 8.5, even more preferably from about 7 to about 8.5, and most preferably from about 7 to about 7.5.

Regardless of the exact pH of the sample mixture or solution, if the polyion is a polycation, the pH may typically be about 0.5 pH units, and more preferably about 1.0 pH unit, less than the pKa of the selected polycation to be bound to the separation media. Accordingly, in one example, the pKa of the polycation is greater than about 8.0, preferably greater than about 9.0, more preferably greater than about 10.0, and most preferably greater than 11.0

Likewise, regardless of the exact pH of the sample mixture or solution, if the polyion is a polyanion, the pH may typically be about 0.5 pH units, and more preferably about 1.0 pH unit, greater than the pKa of the selected polyanion to be bound to the separation media. Accordingly, in one example, the pKa of the polyanion is less than about 8.0, preferably less than about 7.0, more preferably less than about 6.0, still more preferably less than about 5.0 and most preferably less than 4.0.

Washing and Wash Solutions

A wash solution may be used in accordance with the present invention to purify the isolated biological molecule by removing impurities from the separation media before eluting the biological molecule from the separation media. The wash solution may also serve the purpose of disassociating the polyion from both the separation media and the biological molecule, while allowing the biological molecule to remain associated with the separation media.

Any of a number of ionic solutions may be used as the wash solution. Typically, the wash solution may be a salt solution comprising a monovalent salt, a divalent salt, or a combination of both. Preferably, the wash solution comprises a monovalent salt. Exemplary monovalent salts include, for example, lithium fluoride (LiF), lithium chloride (LiCl), lithium bromide (LiBr), lithium iodide (LiI), sodium fluoride (NaF), sodium chloride (NaCl), sodium bromide (NaBr), sodium iodide (NaI), potassium fluoride (KF), potassium chloride (KCl), potassium bromide (KBr), potassium iodide (KI), rubidium fluoride (RbF), rubidium chloride (RbCl), rubidium bromide (RbBr), rubidium iodide (RbI), cesium fluoride (CsF), cesium chloride (CsCl), cesium bromide (CsBr), and cesium iodide (CsI), among others. Preferably, the wash solution will comprise some concentration of a monovalent salt, preferably KCl, and more preferably NaCl.

In certain embodiments, the wash solution may comprise a divalent salt. Exemplary divalent salts include, for example, beryllium chloride (BeCl₂), beryllium fluoride (BeF₂), beryllium bromide (BeBr₂), beryllium iodide (BeI₂), magnesium chloride (MgCl₂), magnesium fluoride (MgF₂), magnesium bromide (MgBr₂), magnesium iodide (MgI₂), calcium chloride (CaCl₂), calcium fluoride (CaF₂), calcium bromide (CaBr₂), calcium iodide (CaI₂), strontium chloride (SrCl₂), strontium fluoride (SrF₂), strontium bromide (SrBr₂), strontium iodide (SrI₂), barium chloride (BaCl₂), barium fluoride (BaF₂), barium bromide (BaBr₂), barium iodide (BaI₂), manganese fluoride (MnF₂), manganese chloride (MnCl₂), manganese bromide (MnBr₂), and manganese iodide (MnI₂), among others. Preferably, the divalent salt is MnCl₂, MgCl₂, or CaCl₂.

Generally, the wash solution may be of any ionic strength. However, selection of the exact ionic strength of the solution is dependent upon a number of factors, including, for example, the polyion used to charge the separation media, the biological molecule that is bound to the polyion and that is to remain associated with the separation media, the strength of the non-covalent bond formed between the separation media and the polyion, and the type and size of the impurity to be removed. Specifically, the ionic strength of the wash solution may be adjusted, for example, to discriminate different sizes of nucleic acids fragments, such as for example, to remove unincorporated primers and primer dimers from PCR samples, while allowing the desired PCR product to remain bound to the separation media. Typically, therefore, the wash solution will be a salt solution having a salt concentration of about 0.1M to about 3M, particularly when the wash solution is devoid of alcohol, preferably from about 0.1M to about 2M, more preferably from about 0.25M to about 2.0M, still more preferably from about 0.25M to about 1M, even more preferably from about 0.5M to about 1M, and most preferably from about 0.75M to about 1M. In a particular embodiment, the wash solution is a salt solution having a salt concentration of about 0.5M. In another embodiment, the wash solution is a salt solution having a salt concentration of about 1M.

Typically, the wash solution will also contain a component or reagent in which the biological molecule is not soluble or which could be used to precipitate the biological molecule. Such a component aids in causing the biological molecule to associate with the separation media in the absence of the polyion by preventing the biological molecule from becoming soluble in the wash solution. This also allows for the use of a wash solution of low ionic strength. When such a component or reagent is present in the wash solution, the concentration of the component or reagent may be adjusted, for example, to discriminate different sizes of nucleic acid fragments, such as for example, to remove unincorporated primers and primer dimers from PCR samples, while allowing the desired PCR product to remain bound to the separation media; in particular, it may be adjusted in the absence of or in lieu of adjusting the ionic strength of the wash solution. Generally, as the concentration of the component or reagent increases, the size of the biological molecule that remains associated with the separation media decreases. Thus, the polyion and selected impurities are removed from both the separation media and the biological molecule, while the biological molecule remains associated with the separation media. Examples of such components include, for example, alcohols, such as, for example, methanol, ethanol, propanol, isopropanol, butanol, and pentanol; non-charged, hydrophilic polymers, such as, for example, polyethylene glycol (PEG), tetraethylene glycol (TEG), polyacrylamide, and glycogen. The wash solution may also contain a composition or reagent which could be used to promote or enhance the removal of the polyion from the separation media. Such a component promotes or enhances the disassociation of the polyion from both the separation media and the biological molecule. The inclusion of such a composition or reagent in a wash solution also allows for the use of a wash solution of low ionic strength. Examples of such components include, for example, polyions having a charge that is opposite that of the polyions used to bind the biological molecule. By way of example, if a polycation is used to bind the biological molecule, a polyanion may be added to the wash solution to promote or enhance the disassociation of the polycation from both the separation media and the biological molecule. Likewise, if a polyanion is used to bind the biological molecule, a polycation may be added to the wash solution to promote or enhance the disassociation of the polyanion from both the separation media and the biological molecule.

Particular compositions or reagents that may be used to promote or enhance the removal of the polyion from the separation media or biological molecule include, for example, chelators. Chelators promote or enhance the removal of the polyion from the separation media by coordinated charge interaction and are of particular use in wash solutions when the separation media being used to bind the biological molecule is an exchange matrix, such as, for example, a chelating or a cation exchange matrix. Examples of useful chelators include, for example, EGTA, EDTA, CDTA.

The wash solution may also contain a metal ion. The presence of a metal ion in the wash solution generally promotes or enhances the removal of the polyion from both the separation media and the biological molecule. This is particularly the case when the separation media is a polyion-charged exchange matrix, such as, for example, a chelating or cation exchange matrix. Although any metal ion may be used in the wash solution, particularly preferred metal ions include multivalent metal ions, and in particular divalent metal ions, such as, for example, those disclosed above. Particularly preferred divalent metal ions include, for example, Mg²⁺, Ca²⁺, and Mn²⁺, typically added to the solution as MgCl₂, CaCl₂, and MnCl₂, respectively.

In order to enhance the efficiency of removal of impurities bound to the separation media, the wash solution may also contain a polyion. The polyion may be either the same or different from the polyion used to bind the biological molecule to the separation media. Typically, the polyion will be present in a concentration of about 1 mM to about 100 mM, preferably about 25 mM to about 75 mM, more preferably about 25 mM to about 50 mM, more preferably about 30 mM, still more preferably about 35 mM, even more preferably about 40 mM, still more preferably about 45 mM, and most preferably about 50 mM. Exemplary polyions and the methods for selecting the same are described above.

Moreover, more than one wash step may be used in accordance with the present method. Additional wash steps may employ the same wash solution or use a wash solution comprising different components or having a different ionic strength. Components that may be used in the additional wash steps and solutions are as described above.

Typically, the first wash solution may be of a higher ionic strength than the subsequent or final wash solution. A first wash solution may have, for example, a salt concentration of about 0.1M to about 3M, preferably from about 0.1M to about 2M, more preferably from about 0.25M to about 2M, and most preferably from about 0.5M to about 1M. Typically, a subsequent or a final wash solution may be of a lower ionic strength than the previous wash solution(s). A subsequent or a final wash solution may have, for example, a salt concentration of about 0 mM to about 200 mM, preferably from about 1 mM to about 200 mM, more preferably from about 1 mM to about 100 mM, still more preferably from about 1 mM to about 50 mM, and most preferably from about 1 mM to about 10 mM.

Accordingly, in one embodiment, the present method utilizes a single wash step with a wash solution of high ionic strength. In another embodiment, the present method utilizes a single wash step with a wash solution of moderate ionic strength. In yet another embodiment, the present method utilizes a single wash step with a wash solution of low ionic strength. In a particularly preferred embodiment, the present method utilizes two wash steps, wherein the first wash step utilizes a wash solution of high or moderate ionic strength and the second wash step utilizes a wash solution of moderate or low ionic strength.

Elution and Elution Solutions

Once washed, the bound biological molecule is typically eluted from the separation media. Conventional elution techniques such as varying the pH, the temperature, the salt or buffer concentrations, competitive binding, and the like may be performed. Particularly preferred elution techniques include the use of an elution solution to remove or desorb the bound biological molecule from the separation media.

Particularly preferred methods of eluting the biological molecule include the use of any number of elution solutions. As discussed above, it has been determined that the use of a polyion non-covalently bound to a separation media to bind a biological molecule allows for the elution of the biological molecule from the separation media using a low ionic strength or nonionic elution solution. Generally, such solutions are compatible with the isolated biological molecule and its intended use and will not, therefore, for example, affect the molecule's ability to subsequently perform its biological function. Because the biological molecule is only loosely associated with or bound to the separation media, the polyion previously binding the biological molecule to the separation media having been removed during the wash step, the biological molecule may be eluted under very mild conditions, and in particular, under conditions that allow for the eluted biological molecule to be further used or processed without additional purification steps, such as, for example, without the removal of high concentrations of salts typically used in an elution step. As such, the biological molecule may be eluted by, for example, very mild solutions, including, for example, nonionic solutions. Examples of suitable mild solutions include, for example, water, and preferably nuclease- and/or protease-free distilled water, or common buffers, such as, for example, Tris, citrate, acetate, phosphate, Tris-EDTA, PBS, Imidazole, and CHES, or reaction mixtures or compositions, such as, for example, a composition for performing PCR, sequencing, or reverse transcription. Accordingly, in one embodiment the elution solution comprises water, and in particular distilled water. In another embodiment, the elution solution comprises a buffer, and in particular PBS, Tris, or a combination of both.

The elution solution may also be any of a number of ionic solutions. Because the biological molecules are very loosely associated with or bound to the separation media after the wash step, the elution solution may be, for example, a low ionic strength salt solution comprising a monovalent salt, a divalent salt, or a combination of both. Examples of suitable monovalent and divalent salts are described above with respect to wash solutions. Preferably, a salt-containing elution solution will contain some concentration of a salt, preferably a monovalent salt, and most preferably NaCl.

Selection of the ionic strength of the solution is dependent upon a number of factors, including, for example, the type and size of the biological molecule that is bound to or associated with the separation media. Typically, mild salt solutions, such as for example, solutions having a salt concentration of less than about 0.5M, preferably less than about 0.3M, more preferably less than about 0.25M, even more preferably less than about 0.2M, still more preferably less than about 0.1M, and most preferably less than about 0.05M may be used. In a particular embodiment, the elution solution is a mild salt solution having a salt concentration of about 0.5M. In another embodiment, the elution solution is a mild salt solution having a salt concentration of about 0.3M. In yet another embodiment, the elution solution is a mild salt solution having a salt concentration of about 0.2M. In still another embodiment, the elution solution is a mild salt solution having a salt concentration of about 0.1M. In a particularly preferred embodiment, the elution solution is a mild salt solution having a salt concentration of less than about 0.1M.

Kits

Another aspect of the present invention is a kit comprising the charged separation media. The kits may be used to bind and optionally isolate a biological molecule according to the methods of the present invention.

The kits of the present invention comprise a separation media, a polyion, and instructions, wherein the polyion is supported or supportable by the separation media by non-covalent bonding and the instructions provide directions for the use of the separation media charged with the polyion. In a particular embodiment of the kit, the separation media is charged with the polyion. The charged separation media comprises a polyion non-covalently bound to the separation media. Suitable separation media, polyions, and methods of charging the separation media are disclosed above.

The instructions or instructional materials contained in the kit generally detail information, including methods, regarding the use of the kit to bind and optionally isolate biological molecules. In particular, the instructions disclose methods of using the kit to bind and optionally isolate biological molecules according to the methods of the present invention as described above.

While the instructions typically comprise written or printed materials, they are not limited to such. Any medium capable of storing such instructions and communicating them to an end user is contemplated. Such media include, but are not limited to electronic storage media (e.g., magnetic discs, tapes, cartridges, chips), optical media (e.g., CD ROM), and the like. Such media may include addresses to internet sites that provide such instructional materials.

The kits may also comprise a wash solution that may be used to remove the polyion but not the biological material from the separation media. Additionally, the kit may comprise an elution solution that may be used to elute the biological molecule from the separation media, and in particular, to elute the biological molecule from the separation media once the polyion has been removed from both the separation media and the biological molecule. Suitable wash and elution solutions are disclosed above.

Having described the invention in detail, it will be apparent that modifications and variations are possible without departing from the scope of the invention defined in the appended claims.

### EXAMPLES

The following non-limiting examples are provided to further illustrate the present invention.

### Example 1

### DNA Binding by Different Separation Media Charged with Spermine

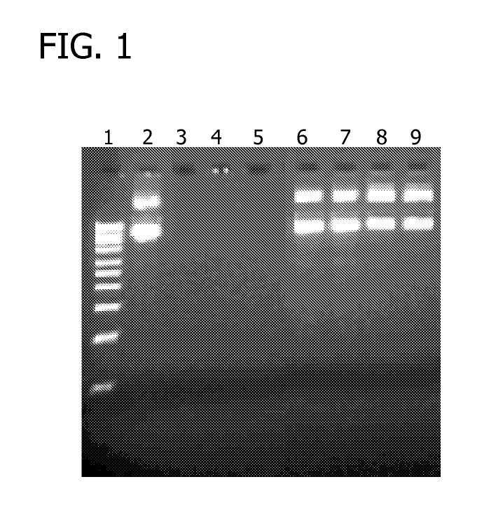

A DNA solution was prepared comprising 50 μg/ml pSPORT-βgal plasmid DNA (7.9 kb), 100 mM NaCl, 50 mM Tris-HCl (pH 8.0). Seven separation media in the form of membrane or filter were each punched into small discs of a quarter inch in diameter. Each disc was placed in a mini spin basket to form a binding column. Each column was then charged by adding either 200 μl of a 0.5M spermine solution or 200 μl of water. The column was centrifuged immediately for 1 minute at maximum speed (14,000 rpm) to remove all liquid from the separation media. DNA binding was performed by adding 100 μl of the DNA solution to each column and centrifuging the column immediately for 1 minute at maximum speed. The flow-through was collected and 3 μl of each sample was resolved on an agarose gel. The results of agarose electrophoresis showed that no DNA was detectable in the flow-through samples from the columns charged with spermine, while the flow-through samples from the columns charged with water contained DNA in nearly 100% proportion of the starting DNA solution. The results demonstrate that negatively charged separation media can bind negatively charged DNA through the polycation linkage of spermine. The results from one of the separation media are shown in FIG. 1. The separation media and their functional groups are shown in Table 1.

TABLE 1
Separation media and functional groups

| | Membrane/ | | Functional |
|---|---|---|---|
| | Filter | Source | group |
| Separation Media | I.C.E. 450 | PALL | Sulfonic acid |
| #1 | | | |
| Separation Media | Biodyne C | PALL | Carboxylic |
| #2 | | | acid |
| Separation Media | Cation Exchange- | 3M Empore | Sulfonic acid |
| #3 | SR | | |
| Separation Media | Chelating | 3M Empore | Iminodiacetic |
| #4 | Sorbent | | acid |
| Separation Media | Vivapure Metal | Vivascience | Iminodiacetic |
| #5 | Chelate | | acid |
| Separation Media | Vivapure C | Vivascience | Carboxylic |
| #6 | | | acid |
| Separation Media | Porex Carboxyl | Porex | Carboxylic |
| #7 | Filter | | acid |

### Example 2

### PCR Cleanup

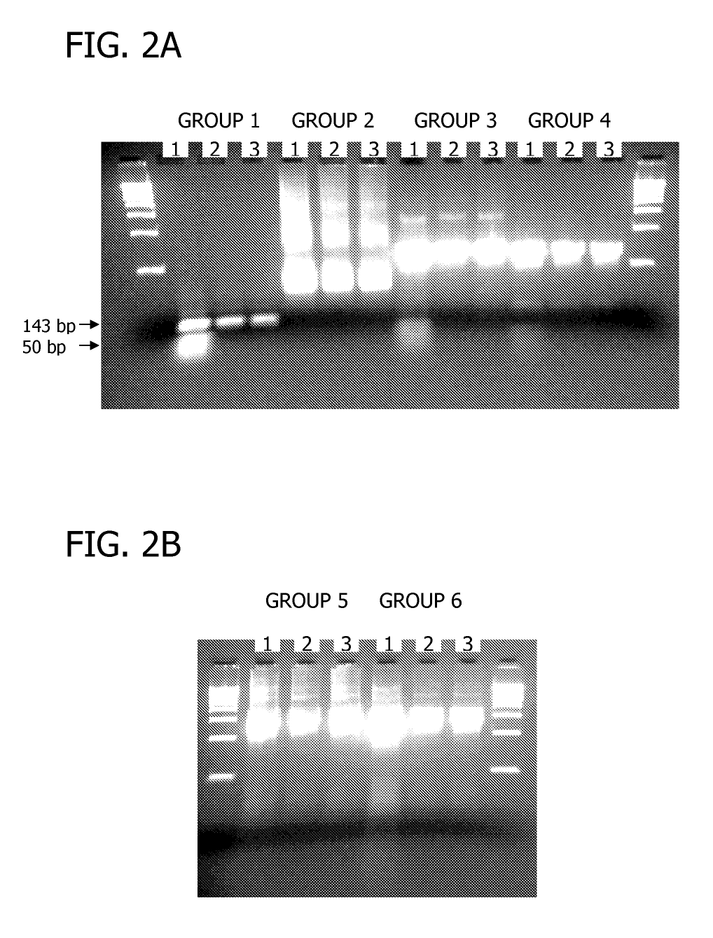

PCR samples were prepared with three different Sigma Taq polymerase blends (Taq (Sigma Product Number D1806); RedTaq® (Sigma Product Number D4309); and AccuTaq™ (Sigma Product Number D8049)). The amplicons ranged from 143 bp to 1.5 kb. Two spermine solutions (0.5M and 1M) were prepared by dissolving spermine powder in water. A salt wash solution and an alcohol wash solution were also prepared. The salt wash solution comprised 2 N NaCl, 10% PEG, and 20% isopropanol, and the alcohol wash solution comprised 80% ethanol and 10 mM tris, pH 8.5. Samples of Sigma's Silica Filter Column G4669 (Sigma-Aldrich Co., St. Louis, Mo.), each consisting of a mini spin basket, 2 layers of Ahlstrom glass filter Grade 121, and 1 layer of Ahlstrom glass filter Grade 141, were used as a purification device. Each column was charged by placing 50 μl of a spermine solution onto the filter and centrifuging for 1 minute at maximum speed in a standard microcentrifuge. Each charged column was then loaded with 50 μl of PCR product and the column was centrifuged 1 minute at maximum speed. Note that the PCR product was loaded directly onto the column and no additional treatment or binding solution was needed. The column was washed twice with 500 μl of the salt wash solution and twice with 500 μl of the alcohol wash solution. Purified DNA was eluted in 50 μl of a tris buffer (10 mM tris, pH 8.5) and analyzed by agarose gel electrophoresis. The results are shown in FIGS. 2A and 2B. As shown in FIGS. 2A and 2B, PCR fragments greater than 143 bp were quantitatively recovered and primer dimmers of about 50 bp were completely eliminated. No significant difference was observed between 0.5M and 1M spermine solutions.

### Example 3

### Purification of Plasmid DNA from Overnight Cultures

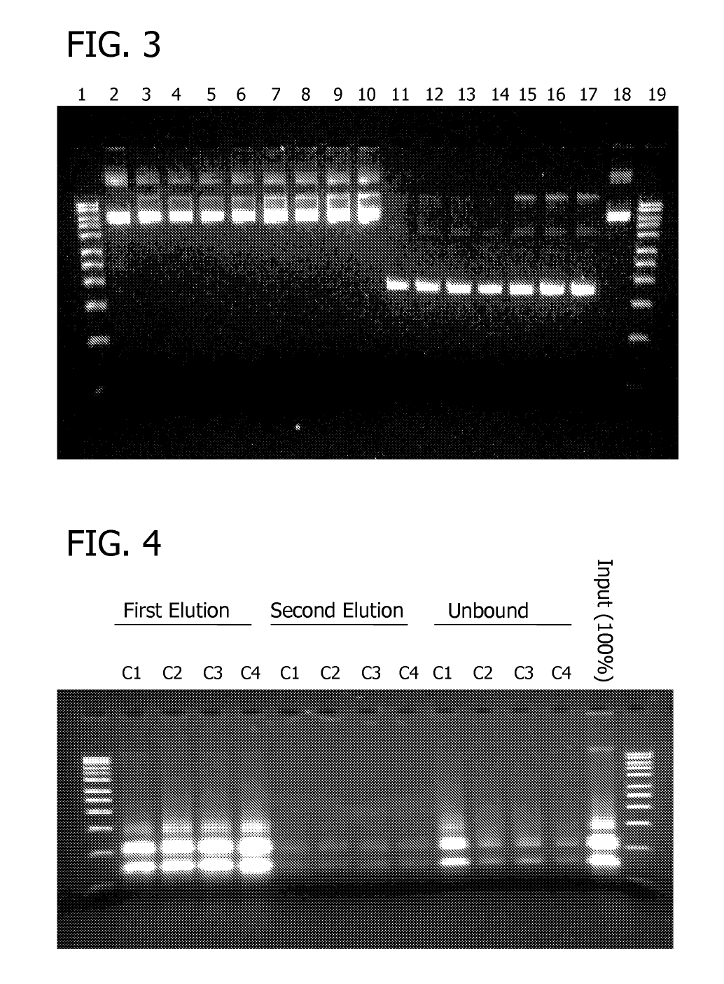

E. coli strains DH5α carrying pSPORT-βgal (7.9 kb) and HB101 carrying Bluescript SK (3.0 kb) were grown overnight in LB broth supplemented with ampicillin (100 μg/ml). Aliquots of the cultures in 400 μl or 800 μl each were directly lysed with 1/10 volume of a lysis solution (20 mg/ml lysozyme, 10 mg/ml RNase A, 5% Triton X-100, 20% glycerol, 200 mM EDTA, 50 mM Tris-HCl, pH 8.0), without removal of the culture medium, for about 3 minutes. Lysates were filtered through a filtration column (one layer of Ahlstrom glass filter paper Grade 121 and one layer of Affco felt filter Grade 8588) by centrifugation for 1 minute at maximum speed. Silica columns (Sigma Product No. G4669, Sigma-Aldrich Co., St. Louis, Mo.) were each charged with 100 μl of a 1M spermine solution as described in Example 1. Each charged column was loaded with 400 μl or 800 μl of cleared lysate, and the column was then centrifuged for 1 minute at maximum speed. Again, no additional treatment or binding solution was added. The column wash and plasmid DNA elution steps were as described in Example 1. Purified plasmid DNA was analyzed by spectrophotometer and by agarose gel electrophoresis. Plasmid DNA recovery for pSPORT-βgal ranged from 6 μg to 7 μg per 400 μl of overnight culture and from 12-14 μg per 800 μl of overnight culture. The recovery for Bluescript SK was about 4 μg per 400 μl of overnight culture and about 7 μg per 800 μl of overnight culture. The A260/A280 purity ratios were all between 1.8 and 1.9. The results are shown in FIG. 3.

### Example 4

### Plasmid Purification with Different Salt Wash Solutions

E. coli strain DH5α carrying pSPORT-βgal (7.9 kb) was grown overnight in LB broth supplemented with ampicillin (100 μg/ml). The overnight culture was directly lysed with 1/10 volume of the lysis solution as described in Example 1. The lysate was clarified by a filtration column (one layer of Ahlstrom glass filter paper Grade 121 and one layer of Affco felt filter Grade 8588). Silica columns (Sigma Product No. G4669, Sigma-Aldrich Co., St. Louis, Mo.) were charged with spermine as described in Example 2. For each assay, each charged column was loaded with 500 μl of clarified lysate, and the column was centrifuged for 1 minute at maximum speed. Each column was then washed with one of several different salt wash solutions, followed by the alcohol wash solution, as described in Example 1. Purified plasmid DNA was eluted in a tris buffer (10 mM tris, pH 8.5) and analyzed by spectrophotometer and agarose gel electrophoresis. The following salt wash solutions were found to be effective for the plasmid purification: 1) 10 μM MnCl₂, 0.5M NaCl, 30% isopropanol; 2) 1M NaCl, 25% isopropanol; 3) 2M NaCl, 25% isopropanol; 4) 1.2M NaCl, 30% isopropanol; and 5) 2.0M NaCl, 30% isopropanol. The results are shown in Table 2.

TABLE 2
Yields of plasmid DNA recovered with different salt
wash solutions

| Wash solution | Yield (μg) | A₂₆₀/A₂₈₀ ratio |
|---|---|---|
| 10 μM MnCl₂, 0.5M NaCl, 30% | 10.8 | 1.84 |
| isopropanol | | |
| 1M NaCl, 25% isopropanol | 12.1 | 1.88 |
| 2M NaCl, 25% isopropanol | 10.9 | 1.9 |
| 1.2M NaCl, 30% isopropanol | 12.6 | 1.91 |
| 2.0M NaCl, 30% isopropanol | 12 | 1.9 |

### Example 5

### RNA Capture and Recovery

Four different silica columns were assembled with the following filter combinations: Column #1: 2 layers of Osmonics glass filter paper G15; Column #2: 1 layer of G15 and 1 layer of Ahlstrom glass filter paper Grade 151; Column #3: 1 layer of G15 and 1 layer Ahlstrom glass filter paper Grade 121; and Column #4: 1 layer of Grade 151 and 1 layer of Grade 121. Each column was charged with 100 μl of a 0.5M spermine solution as described in Example 1. For each assay, each charged column was loaded with 500 μl of a solution (100 mM NaCl, 50 mM Tris-HCl, pH 7.4) containing 25 μg of S. cerevisiae total RNA and the column was centrifuged for 1 minute at maximum speed. RNA in the flow through fraction was also analyzed by spectrophotometer and agarose gel electrophoresis. The column wash steps were as described in Example 1. Captured RNA was eluted twice, each in 50 μl RNase-free water and analyzed by spectrophotometer and agarose gel electrophoresis. RNA recovery was about 50% for Column #1, about 70% for Columns #2 and #3, and about 90% for Column #4. The integrity of recovered RNA samples was the same as that of the starting material by agarose gel electrophoresis analysis. The results are shown in FIG. 3.

When introducing elements of the present invention or the preferred embodiments(s) thereof, the articles “a”, “an”, “the”, and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions, kits, and methods without departing from the scope of the invention, it is intended that all matter contained in the above description shall be interpreted as illustrative and not in a limiting sense.

1-32. (canceled)

33. A method of binding and optionally isolating a biological molecule from a mixture, the method comprising combining the mixture with a separation media having a polyion non-covalently bound to the surface thereof to bind the biological molecule to the separation media.

34. The method of claim 33, wherein said method further comprises washing the separation media with a wash solution and eluting the biological molecule from the separation media with an elution solution.

35. The method of claim 34, wherein the wash solution is capable of removing impurities bound to the polyion.

36. The method of claim 34, wherein the wash solution is capable of removing the polyion but not the biological molecule from the separation media.

37. The method of claim 33, wherein said method further comprises washing the separation media with a first wash solution to remove impurities bound to the polyion, washing the separation media with a second wash solution to remove the polyion but not the biological molecule from the separation media, and eluting the biological molecule from the separation media with an elution solution.

38. The method of claim 34, wherein the separation media is selected from the group consisting of a chelating matrix, a cation exchange matrix, an anion exchange matrix, and a silica matrix.

39. The method of claim 38, wherein the matrix is selected from the group consisting of resins, filters, beads, membranes, and coated surfaces.

40. The method of claim 34, wherein the polyion is a polyanion.

41. The method of claim 40, wherein the polyanion is selected from the group consisting of a carboxylic acid, a polypeptide, or a polysaccharide.

42. The method of claim 41, wherein the polyanion is a carboxylic acid selected from the group consisting of citric acid, succinic acid, and polyacrylic acid, a polypeptide selected from the group consisting of polyglutamic acid and polyaspartic acid, or the polysaccharide heparin sulfate.

43. The method of claim 34, wherein the polyion is a polycation.

44. The method of claim 43, wherein the polycation is a polyamine or a polypeptide.

45. The method of claim 44, wherein the polycation is a polyamine selected from the group consisting of spermine, spermidine, and putrescine or a polypeptide selected from the group consisting of polylysine, polyarginine, and polyhistidine.

46. The method of claim 34, wherein the biological molecule is selected from the group consisting of a nucleic acid, a modified nucleic acid, and a protein.

47. The method of claim 46, wherein the biological molecule is a nucleic acid selected from the group consisting of DNA and RNA or a modified nucleic acid selected from the group consisting of PNA or LNA.

48. The method of claim 34, wherein the wash solution comprises a salt and an alcohol.

49. The method of claim 48, wherein the salt is present in a concentration of about 0.1M to about 2.0M.

50. The method of claim 49, wherein the salt is selected from the group consisting of NaCl, KCL, MgCl₂, MnCl₂, and CaCl₂.

51. The method of claim 48, wherein the alcohol is present in a concentration of about 10% wt/v to about 80% wt/v.

52. The method of claim 51, wherein the alcohol is selected from the group consisting of methanol, ethanol, propanol, isopropanol, butanol, and pentanol.

53. The method of claim 48, wherein the wash solution further comprises a chelator, a hydrophilic non-charged polymer, or a combination of both.

54. The method of claim 53, wherein the hydrophilic, non-charged polymer is selected from the group consisting of polyethylene glycol, tetraethylene glycol, glycogen, and polyacrylamide.

55. The method of claim 48, wherein the elution solution is selected from the group consisting of water, a buffer, a salt solution, or a reaction composition.

56. The method of claim 55, wherein the elution solution is water.
